(19) United States
(12) Patent Application Publication
(10) Pub. No.: US 2011/0257378 A1
(43) Pub. Date: Oct. 20, 2011

(54) AQUEOUS TWO PHASE EXTRACTION AUGMENTED PRECIPITATION PROCESS FOR PURIFICATION OF THERAPEUTIC PROTEINS

(75) Inventors: Richard Tran, London (GB); Nigel J. Titchener-Hooker, London (GB); Karol Lacki, Uppsala (GB)

(73) Assignee: GE HEALTHCARE BIO-SCIENCES AB, Uppsala (SE)

(21) Appl. No.: 13/131,034

(22) PCT Filed: Nov. 18, 2009
(86) PCT No.: PCT/SE2009/051305

§ 371 Date: Jun. 14, 2011

(30) Foreign Application Priority Data
Nov. 25, 2008 (SE) 0802477-0

(51) Int. Cl. C07K 1/30; C07K 1/36

(52) U.S. Cl. 530/421

(57) ABSTRACT

The invention relates to an aqueous two phase extraction (ATPE) augmented precipitation process, which may be used to recover and also partially purify therapeutic proteins, including monoclonal antibodies from a crude multi-component mixture. The process involves the formation of a forward extraction PEG-Phosphate ATPE system in which the target product is preferentially partitioned to the polymer rich phase. A second ATPE back extraction system is then formed by introducing the polymer rich phase from the forward extraction to a new phosphate salt rich phase, causing the product to precipitate at the interface between the two phases. This precipitate is then recovered and resolubilised in a suitable buffer and may be passed on for further purification.

### CROSS-REFERENCE TO RELATED APPLICATIONS

This application is a filing under 35 U.S.C. §371 and claims priority to international patent application number PCT/SE2009/051305 filed Nov. 18, 2009, published on Jun. 3, 2010 as WO 2010/062244, which claims priority to application number 0802477-0 filed in Sweden on Nov. 25, 2008.

### FIELD OF THE INVENTION

The present invention relates to the field of protein purification and in particular, methods for capturing and purifying proteins from crude multi-component mixtures. Specifically, it relates to the use of a combination of an aqueous two phase extraction system and a protein precipitation process, in order to affect bioseparation of the target molecule.

### BACKGROUND OF THE INVENTION

Monoclonal antibodies (mAbs) currently represent the most prevalent biopharmaceutical product in either manufacture or development by organisations worldwide (see Jacobi A, Eckermann C and Ambrosius, “Bioseparation and Bioprocessing” 2^{nd} Edition Volume 2 2007 Wiley-VCH p 431). The high commercial demand for and hence value of this particular therapeutic market has lead to the emphasis being placed on pharmaceutical companies to maximise the productivity of their respective mAb manufacturing processes whilst controlling the associated costs.

Technological developments upstream have gone some way towards addressing this challenge with advances in mammalian cell culture technology resulting in typical mAb titres rising from tenths of a gram per liter, up to 10 grams per liter, over the past decade (see Thömmes J, Etzel M. “Alternatives to Chromatographic Separations”, Biotechnology Progress 2007; 23: 42-45). Along with these higher product concentrations, advances in bioreactor technology have meant that companies now operate cell culture processes with volumes of up to 25,000 L (see Kelley B. “Very Large Scale Monoclonal Antibody Purification: The Case for Conventional Unit Operation”, Biotechnology Progress 2008; 23: 995-1008). Whilst upstream operations have advanced in order to meet the challenges of increasing product demand, it has been argued that the to developments in downstream processing (DSP) technology have not kept pace. The downstream purification of the majority of mAb products, either marketed or in development, is currently based on the utilisation of process platform built around the use of Protein A affinity chromatography (see Shukla A A, Hubbard B, Tressel T, Guhan S, Low D. “Downstream processing of monoclonal antibodies—Application of platform approaches”, Journal of Chromatography B 848: 28-39 and see Sommerfeld S and Strube J. “Challenges in biotechnology production—generic processes and process optimisation for monoclonal antibodies”, Chemical Engineering and Processing 2005; 44: 1123-1137). This heavy dependency upon the use of Protein A for mAb purification has been the cause for some concern amongst process engineers involved in the purification of mAb products.

Protein A chromatography processes are scaled based upon the mass of product which needs to be captured. A 10,000 L fermenter may be used to culture a cell line expressing mAb at a concentration of 1 g/L, thereby producing a total of 10 kg of mAb per batch. If the same fermenter is used to culture a cell line expressing mAb at a concentration of 10 g/L, the volume of the cell culture will still be 10,000 L, but the mass of mAb contained within will now be 100 kg. Thus the Protein A chromatography column will need to be either made 10 times as large as that which was used to capture the 10 kg batch (which may not be possible due to plant space limitations) or instead cycled a greater number of times per batch which will increase the process time. As a result chromatography effectively imposes a constraint upon the maximum productivity of a biopharmaceutical manufacturing facility. Given the present market for mAb therapeutics, cases in which these constraints have been reached are rare (see Kelley B. “Very Large Scale Monoclonal Antibody Purification: The Case for Conventional Unit Operation”. Biotechnology Progress 2008; 23: 995-1008). However, in the face of increasing product demand, some engineers have questioned the long term sustainability of the current paradigm for mAb purification. Companies now have an ever increasing portfolio of mAb candidates in the development pipeline. Even if only a fraction of these candidates make it through clinical trials, this will still lead to the need for multi-product facilities, operating very quick, intensive manufacturing campaigns which may challenge productivity constraints. Additionally, mAbs products are finding applications beyond those for which they were originally intended. This could dramatically increase the demand for a particular mAb which may again push plant productivities beyond their limit.

In light of this, process engineers have begun investigating what may be termed alternative bioseparation technologies, which have the potential to offer higher processing capacities, as well as possibly providing better economies of scale than packed bed chromatography (see Przybycien, T. M.; Pujar, N. S.; Steele, L. M. “Alternative bioseparation operations: life beyond packed-bed chromatography” Current Opinion in Biotechnology 2004, 15 (5), 469-478). Two such separation techniques are aqueous two phase extraction (ATPE) and precipitation. These have attracted interest due to their relatively low associated operating costs and ease of scaling. Also because both are based on bulk mixing of the process fluid, these techniques will scale with the volume of the process stream, rather than the mass of product contained within, as is the case with packed bed chromatography. For example a 10,000 L cell culture would require a 10,000 L precipitation tank, regardless of whether the cell culture produced mAb at a concentration of 1 g/L or 10 g/L.

A drawback of precipitation processes is the relatively low purification factors achievable. This is down to not only the non-specific mechanism of separation, but also the potential for impurity entrapment within the precipitate complex, leading to the need for extensive precipitate washing prior to resolubilisation in order to maximise product purity. Process robustness is another issue, with screening of a wide range of conditions necessary to determine the optimal operating parameters for each new antibody product.

Likewise aqueous two phase extraction (ATPE) has also been found to suffer some drawbacks. Several studies have been performed in which aqueous two phase extraction processes have been optimised for the purification of monoclonal antibodies (see Andrews B A, Nielsen S, Asenjo J A. “Partitioning and purification of monoclonal antibodies in aqueous two-phase systems”, Bioseparation 1996; 6: 303-313. and Azevedo A M, Rosa P A J, Ferreira I F, Aires-Banos M R. “Optimisation of aqueous two-phase extraction of human antibodies”, Journal of Biotechnology 2007; 132: 209-217). These studies showed relatively promising results, with high antibody yields achieved, however only modest purification factors were obtained due to the non-specific mechanism of separation employed by ATPE processes.

Key processing parameters associated with ATPE are all concerned with affecting the partitioning behaviour of the process stream components. Whether a molecule partitions into the top or bottom phase will be determined by the properties of the molecules (e.g. charge, MW and solvation) as well as those of the polymer (e.g. conc., MW, hydrophobicity). The physicochemical environment (e.g. temperature, pH and ionic strength) of the system will also influence partitioning behaviour. Due to the various interacting factors and the careful balance required between different operating parameters to ensure optimal performance, ATPE systems can be relatively non-robust. The situation is further exacerbated by the lack of fundamental knowledge regarding the partitioning of biological components in aqueous two phase systems. Process optimisation therefore requires a screening of a wide range of conditions along with the adoption of a design of experiments approach. As a result, for a new antibody product, extensive process development of an ATPE operation is not only a very necessary endeavour, but it is also a lengthy one. This compares unfavourably with Protein A affinity chromatography which displays a high level of robustness and only requires fine tuning of operating conditions in order to achieve optimal performance for a new antibody product.

The partitioning behaviour of different aqueous two phase systems, in terms of phase volume ratios, can be affected not only by the choice of phase forming components and their concentration within the system, but also upon the properties of the feed material. Product concentration is a key aim for any bioseparation technique to be employed early on in a downstream process. Skewed phase volume ratios can adversely affect the concentrating power of ATPE since antibodies may partition to the high volume phase.

Recently several patent applications have been published, regarding the use of precipitation for the purification of protein therapeutics. These are:
Coffman et al. “Separation Methods” (US 2007/0066806);
Farhner et al. “Polyelectrolyte Precipitation and Purification of Proteins” (US 2008/0193981);
Ramanan et al. “Method of Isolating Antibodies by Precipitation” (WO 2008/100578); and
Moya et al. “Purification of Proteins” (WO 2008/079302).

Whilst all aforementioned patent applications are concerned with the purification of monoclonal antibodies (mAb), there are differences in the specific route through which this purification is achieved. WO 2008/079302 and US 2007/0066806 are both concerned with precipitation of impurities in the process stream rather than the target antibody. Removal of the precipitated impurities then allows for purification of the mAb. The precipitation processes described by WO 2008/100578 and US 2008/0193981 can be applied to the precipitation of either target antibody product or impurities in the process stream.

The precipitation processes described by these applications can all be used to process crude fermentation broth containing whole cells. The methods described in WO 2008/100578, US 2008/0193981 and WO 2008/079302 may only be used in this manner if the component(s) being precipitated are process stream impurities. The precipitation process described by US 2007/0066806 can only be used to precipitate impurities.

These applications describe methods which utilise different precipitating agents. US 2008/0193981 describes a method of precipitation which utilises polyelectrolytes that can interact with the target molecule allowing for selective precipitation. US 2007/0066806 utilises a combination of soluble salts which react with one another, when in solution together, to form insoluble salt precipitates. These precipitates associate (during and after formation) with impurities in the process stream allowing for selective precipitation. WO 2008/100578 utilises soluble polymers which have an affinity for the target molecule. Introduction of physicochemical stimuli (e.g. changes in temperature, pH, ionic strength etc.) can cause this polymer to come out of solution and form a precipitate, bringing the target molecule with it. WO 2008/079302 utilises a combination of isoelectric precipitation and also PEG as an additional precipitant. The process must also be performed at relatively low temperatures (2-8° C.) in order to reduce further the solubility of the target molecule.

There remains a need for a separation technique which is considerably more robust and which achieves comparable performance across significantly different feed materials containing different therapeutic proteins of interest.

### SUMMARY OF THE INVENTION

A process for the purification of proteins is provided. This process is based on a polymer-salt Aqueous Two Phase Extraction (ATPE) performed on a multi-component mixture (i.e., the feed) containing the target protein, ultimately resulting in precipitation of the product which may be re-solubilised and passed on for further purification.

The novel ATPE-precipitation process is comprised of two discrete stages. The first forward extraction stage involves introducing the phase forming components (such as Polyethylene Glycol (PEG), Phosphate and NaCl) to the feed causing the formation of a polymer-salt aqueous two phase system in which the target protein preferentially partitions to the polymer rich phase, whilst some impurities move either into the salt rich phase or collect in the form of a precipitate at the interface between the two phases. In the second back extraction stage, the polymer rich phase from the forward extraction is recovered and contacted with a back extraction buffer (for example a phosphate buffer), forming a second aqueous two phase system and in turn causing the target protein to move out of the polymer rich phase and collecting in the form of an interfacial precipitate. This product containing precipitate is recovered using a filter or a centrifuge and then resolubilised in an appropriate re-solubilisation buffer allowing for further processing.

In cases which the process was used for the primary capture of monoclonal antibodies (mAbs) from mammalian cell culture supernatants, yields from this ATPE assisted precipitation process were found to be comparable to yields obtained using protein A chromatography, across a range of different mAbs and feeds. This process thus offers a potential alternative for the primary capture of mAbs from cell culture supernatant.

This method overcomes the drawbacks associated with precipitation and ATPE processes individually by integrating a precipitation process with a two-stage ATPE process. The first forward extraction stage of the ATPE process, allows partial purification of the product mAb, as it preferentially partitions to the polymer rich phase. The second back extraction process, performed on the polymer rich phase recovered from the forward extraction allows for further purification as impurities partition either to the top or the bottom phase, whilst the product mAb precipitates at the interface between the two phases. A combination of purification mechanisms allows for a product which is not only purer than that which may typically be obtained with precipitation or ATPE individually, but also can be obtained in a much more concentrated form than that which might be obtained from a typical ATPE process alone. The combination of ATPE and precipitation has also yielded a separation technique which is considerably more robust, showing comparable performance across significantly different feed materials containing different target proteins.

### BRIEF DESCRIPTION OF THE DRAWINGS

FIG. 1 shows the process scheme for a PEG-phosphate forward extraction aqueous two phase system at either preparative or manufacturing scale, being used to capture a mAb from a mammalian cell culture.

FIG. 2 shows the process scheme for a PEG-phosphate back extraction aqueous two phase system at either preparative or manufacturing scale, being used to capture a mAb from a mammalian cell culture.

FIG. 3 shows a process scheme for mAb precipitate recovery and resolubilisation at either preparative or manufacturing scale.

FIG. 4 shows an overall process flow sheet illustrating the possible equipment requirements for this ATPE augmented precipitation process at either preparative or manufacturing scale.

FIG. 5 is a schematic showing a monoclonal antibody purification process flow incorporating certain embodiments of the invention. This diagram serves to illustrate the ways in which the method described may be employed from a whole bioprocess perspective.

FIG. 6 shows a comparison of Chromatograms from Protein A analyses of top and bottom phases of a forward extraction aqueous two phase system applied to a cell culture feed supernatant containing antibody A and denoted “cell culture supernatant feed A”.

FIG. 7 shows a comparison of Chromatograms from Protein A analyses of top and bottom phases of a forward extraction aqueous two phase system applied to a cell culture feed supernatant containing antibody B and denoted “cell culture supernatant feed B”.

FIG. 8 shows a comparison of Chromatograms from Protein A analyses of top and bottom phases of a back extraction aqueous two phase system performed on the top phase obtained from the forward extraction on cell culture supernatant feed A.

FIG. 9 shows a comparison of Chromatograms, similar to FIG. 8, of top and bottom phases of a back extraction aqueous two phase system performed on the top phase obtained from the forward extraction on cell culture supernatant feed B.

FIG. 10 shows a comparison of Chromatograms from Protein A analyses of the top phase obtained from the forward extraction performed on cell culture supernatant feed B, containing antibody B and of the resolubilised precipitate formed in and recovered from the back extraction aqueous two phase system.

FIG. 11 shows a comparison of Chromatograms from size exclusion chromatography analyses of the cell culture supernatant feed B, containing antibody B, the top phase from the forward extraction aqueous two phase system performed on cell culture supernatant feed B and the resolubilised precipitate subsequently formed in the back extraction aqueous two phase system.

### DETAILED DESCRIPTION OF THE INVENTION

### Definitions

Terms used in the description of the invention are collected and defined.

The term “target molecule”, “target protein” and “protein product” refers to the protein which it is the aim of the method, to cause precipitation of. The protein includes both therapeutic protein and antibody.

The term “multi-component mixture” refers to any aqueous mixture containing more than one type of biological or organic molecule including, recombinant proteins, native host cell proteins, DNA, RNA, viruses and lipids. Aqueous mixtures encompassed by the term “multi-component mixture”, may also contain unlysed whole cells of various types including mammalian, microbial and yeast. The term also covers aqueous mixtures containing fragments of cells, resulting from the lysing and/or homogenisation of mammalian, microbial and yeast cells. The term “multi-component mixture” specifically encompasses mammalian cell culture supernatant and clarified microbial fermentation broth. The term also encompasses clarified microbial lysate and homogenate, blood and blood fractions, and partially purified variants of all multi-component mixtures which have been herein defined to be specifically encompassed by the term.

The term “antibody” means any recombinant of naturally occurring intact antibody, e.g. an antibody comprising an antigen-binding variable region as well as a light chain constant domain (CL) and heavy chain constant domains. Also encompassed by the term are antibody fragments, or molecules including antibody fragments, including, but not limited to, Fab, Fab′, F(ab′)2, Fv and Fc fragments. The term “antibody” specifically encompasses fusion proteins such as Fc fusion proteins, peptibodies and other chimeric antibodies. The term “antibody” specifically encompasses both monoclonal and polyclonal antibodies.

The term “cell culture supernatant” refers to cell culture media from which whole cells have been removed by, for example, filtration. Cell culture supernatant can be but need not be clarified. For the purpose of the invention, the cell culture supernatant is one form of a multi-component mixture which contains the target protein of interest.

The term “host cell proteins” refers to all proteins expressed by the cultured host cell, aside from the protein product, during the course of the cell culture process.

The term “aforementioned patents” refers to US 2007/0066806 (Coffman et al.), US 2008/0193981 (Farhner et al.), WO 2008/100578 (Ramanan et al.) and WO 2008/079302 (Moya et al.)

The term “aqueous two phase system” refers to an aqueous mixture composed of two water-based immiscible aqueous solutions.

The term “incompatible anion” refers to the anion of salts which when in solution with certain polymers, will cause the solution to separate, forming two discreet phases; a polymer rich phase and a salt rich phase. Anions encompassed by the term “incompatible anion” include kosmotropic anions such as phosphate (PO4³⁻), citrate (C₃H₅O(COO)₃³⁻) and sulphate (SO₄²⁻).

The term “phosphate” unless the context clearly dictates otherwise, or it is explicitly stated otherwise, refers to a salt of phosphoric acid, for example sodium phosphate, rather than the phosphate ion (PO₄³⁻).

The term “polymer rich phase”, refers to the phase of an aqueous two phase system, which contains the highest concentration of polymer or polymers.

The term “salt rich phase”, refers to the phase of an aqueous two phase system, which contains the highest concentration of the incompatible anion of the salt used to form the two phase system.

The term “top phase”, refers to the less dense phase of an aqueous two phase system which collects above the bottom phase and also any interfacial precipitate which may have formed in an aqueous two phase system when either the two phase system is left to settle under the influence of gravity, or if the settling is assisted, for example through the use of centrifugation.

The term “bottom phase” refers to the more dense phase of an aqueous two phase system which collects below the top phase and also any interfacial precipitate which may have formed in an aqueous two phase system when either the two phase system is left to settle under the influence of gravity, or if the settling is assisted, for example through the use of centrifugation

The term “interfacial precipitate” refers to precipitate which forms in an aqueous two phase system and which collects at the interface between the top and bottom phases when either the two phase system is left to settle under the influence of gravity, or if the settling is assisted, for example through the use of centrifugation.

### Method for Purification of a Target Protein

The method provided herein is an integrated two step ATPE assisted precipitation process in which the first, so called forward extraction ATPE step removes impurities via preferential partitioning of said impurities to the salt rich phase of the two phase system, as well as causing the precipitation of some impurities, while the second, so called back extraction ATPE step precipitates the product. It is integrated because the conditions of the process stream following the forward extraction step directly prepare it for the second, back extraction step.

In certain embodiments the target protein can be an antibody. The antibody can be either a monoclonal or polyclonal antibody. The antibody can also be an IgG antibody, for example an IgG1, IgG2, IgG3 or IgG4 antibody. Also encompassed by the term antibody are antibody fragments, chimeric antibodies, fusion proteins such as Fc fusion proteins and peptibodies.

In other embodiments, the target protein could be a recombinant protein such as recombinant human growth hormone, recombinant human insulin or interferon. The target protein could also be an enzyme, either in recombinant or native form. In further embodiments the target protein could also be a blood factor.

The purification process described herein can be applied to any multi-component mixture, in which the aim is to isolate and purify a protein product within the mixture from other components, which may include, but is not limited to, native host cell proteins, DNA, RNA, viruses and lipids. In one embodiment of this present invention, the multi-component mixture (i.e., the feed material of the purification process) is a cell culture supernatant, generated through the culture of mammalian cells expressing and secreting an antibody of interest into the culture media. The cell culture supernatant may be obtained by either filtration or centrifugation of the cell culture broth, allowing for removal of whole cells. The feed to the purification process should therefore be preferably free of unlysed whole cells. The cell culture supernatant, however, need not be clarified. Feed material containing whole unlysed cells may be used, providing the composition and conditions of the ATPE forward extraction system cause the antibody product to preferentially partition into the top phase, since in such a system whole cells will move into the bottom phase, The feed material may also contain large cell fragments such as cell debris, which will either partition to the bottom phase or precipitate at the interface during the forward extraction. The optional clarification of the cell culture supernatant may be accomplished using any conveniently available method, for example microfiltration or depth filtration.

The aqueous two phase extraction assisted precipitation purification method described herein is comprised of three discrete stages.

1. Forward extraction
2. Back extraction
3. Precipitate recovery and resolubilisation

### 1. Forward Extraction

The first forward extraction stage involves forming a polymer-salt aqueous two phase system in which conditions are such that the target protein (e.g., antibody) preferably partitions into the polymer rich phase. The two phase system may be formed by adding appropriate amounts of phase forming components, to the feed. Phase forming components should include at least one water soluble polymer, a soluble salt with an anion which, when in solution is incompatible with the polymer and therefore capable of forming an aqueous two phase system with it, and another soluble salt which is used to mediate the partitioning of components in the two phase system. The water soluble polymer may be selected from a list including but not limited to Polyethylene glycol (PEG) or ethylene oxide-propylene oxide (EOPO). The incompatible salt should contain a strongly hydrated anion, and may be selected from a list including but not limited to citrate, phosphate or sulphate. The partition mediating salt should contain a less hydrated anion and may be selected from a list including but not limited to chloride, iodide or nitrate.

In one embodiment, the polymer is a polyethylene glycol (PEG). The PEG can have a molecular weight of between 1,450 Da and 6,000 Da, for example 1,500 Da. The incompatible salt is a mixture of monobasic and dibasic sodium phosphate salt and the partitioning mediating salt is sodium chloride (NaCl). In another embodiment, the polymer is PEG with a molecular weight of 4000 Da, the incompatible salt is sodium citrate and the partition mediating salt is potassium iodide (KI)

In one embodiment of the invention the phase forming components are added to the feed in the form of powders. In another embodiment the phase forming components are added to the feed in the form of concentrated stock solutions. In another embodiment, some of the phase forming components are added in the form of powders whilst others are added in the form of concentrated stock solutions. For example in a PEG-Phosphate ATPE system, with NaCl as the partition mediating salt, the PEG and the phosphate may be added to the feed material in the form of concentrated stock solutions, whilst the NaCl is added in powder form.

The phase forming components should be added in such relative quantities so as to cause the formation of an aqueous two phase system, within which components in the feed display the desired partitioning behaviour. Suitable system compositions may be found in the extensive study performed by Albertsson et al., (see Albertsson, P.-\acute{Å}., 1986. Partition of Cell Particles and Macromolecules, third edition. Wiley, N.Y.). One of ordinary skill in the art will recognise the need to optimise the relative concentration of the phase forming components to not only provide desirable partitioning behaviour during the forward extraction, but also so as to result in a polymer rich phase which is amenable to the back extraction process, and the precipitation of the product protein caused therein.

In one embodiment PEG, phosphate and NaCl are added so as to result in a final system composition of 12%-20% (w/w) PEG, 9%-19% (w/w) Phosphate and 4%-12% (w/w) NaCl. In another embodiment PEG, a mixture of monobasic and dibasic phosphate and NaCl are added so as to result in a final system composition of 15% (w/w) PEG, 14% (w/w) phosphate and 12% (w/w) NaCl. The monobasic phosphate added may be monobasic sodium phosphate (NaH₂PO₄) or monobasic potassium phosphate (KH₂PO₄). The dibasic phosphate used may be dibasic potassium phosphate (K₂HPO₄) or dibasic sodium phosphate (Na₂HPO₄). In determining the amount of phosphate salt to be added to such a system, the mass composition of phosphate should include not only the weight of the phosphate ion, but also the cation of the salt. For example in forming a system with a final overall mass of 30 grams, with the phosphate added in the form of a powder, 4.2 g of anhydrous monobasic sodium phosphate (NaH₂PO₄) powder should be added, in order to give a final Phosphate concentration of 14% (w/w), even though the mass fraction of phosphate (PO₄³⁻) in monobasic sodium phosphate (NaH₂PO₄) means that only 3.325 g of phosphate has been added which corresponds to a PO₄³⁻ concentration of approximately 11.1% (w/w). Hydrated salt powders may be used, for example monobasic sodium phosphate monohydrate (NaH₂PO₄.H₂O), but the mass contribution of water must be accounted for. Similarly is the case for when adding phosphate in the form of a concentrated stock solution.

The phase forming components should preferably be added sequentially, with each component allowed to fully dissolve (when added in the form of a powder) and/or disperse (when added in the form of a concentrated stock solution) under mixing of the bulk fluid, prior to the next component being added. The phase forming components may be added in any order. The components can all be added at once, however this may have undesirable results such as excessive precipitation and longer salt and polymer dissolution and/or dispersion times, caused by poor fluid mixing as a result of increased fluid viscosity. The dissolution/dispersion of phase forming components may be performed at room temperature and pressure, although this may be performed at any temperature which is found to be conducive towards the dissolution/dispersion of phase forming components, for example some Polyethylene glycols are found to be more soluble at lower temperatures.

The system pH should be at least 1 pH unit below the pI of the target protein or antibody in order to ensure the target protein is positively charged. This will not only insure against premature precipitation, but also help partitioning of the target protein to the polymer rich phase of the two phase system.

In an embodiment of the invention in which a mixture of monobasic and dibasic phosphate is used, the system pH may be established by altering the ratio between the amount of monobasic and dibasic phosphate salt added to form the two phase system. Increasing the amount of monobasic phosphate used, whilst decreasing the amount of dibasic phosphate added will result in a lower system pH, while increasing the amount of dibasic phosphate added and decreasing the amount of monobasic phosphate used will increase the pH of the system. The pH of the forward extraction system may be between pH 3.0 and pH 9.0, although a relatively neutral pH is preferable, for example pH 5.0 to 7.0, such as pH 6.0.

Following complete dissolution and/or dispersion of the phase forming components, the forward extraction system should be left to mix for between 10 minutes and 1 hour, for example 30 minutes, before being incubated at room temperature for between 10 minutes to 24 hours, for example 30 minutes. The mixing period following complete powder dissolution and/or stock solution dispersion, is to firstly maximise the surface area for mass transfer between phases, and also to ensure equilibrium is reached with regards to partitioning of components between the polymer rich phase and the salt rich phase. The incubation period is to allow for phase separation under gravity. Dependent upon the complexity of the feed, the presence of precipitate and hence the resultant viscosity of the two phase system, complete phase separation may be accomplished in this manner. In an embodiment in which the present invention is used to purify an antibody product from a cell culture supernatant, the complexity of the cell culture supernatant will mean that this eventuality is unlikely due to an increase in system viscosity, caused by the precipitation of feed components during the forward extraction. Instead complete phase separation and recovery of the polymer rich phase may be accomplished using any conveniently available method, for example centrifugation. Any precipitate formed during the forward extraction, which will settle (possibly requiring some assistance, for example using centrifugation) at the interface between the top and bottom phase. This precipitate will be composed mostly of impurities and contain negligible amounts of target protein and as such its recovery is not required.

In the forward extraction, a combination between salting out effects and electrostatic interactions will cause the target protein to preferably partition into the polymer rich phase. The partition coefficient (top phase concentration/bottom phase concentration), denoted K, of the target protein should be between 5 and >100, for example K=50.

In one embodiment of this invention, the forward extraction process may be performed in a single stirred vessel. The multi-component mixture (e.g., cell culture supernatant) may be held in an agitated vessel to which the phase forming components, PEG, phosphate and NaCl in powder form, are directly and sequentially added. The contents of this vessel may be mixed until all phase forming components are completely dissolved. Further mixing may be performed in order to ensure equilibrium is reached and that complete partitioning of components has occurred. One of ordinary skill in the art will recognise the need to optimise the mixing process, in order to minimise mixing times within the stirred vessel. Optimisation of the mixing process will need to account for, among other things, factors such as the design of agitator(s), vessel dimensions and presence of baffles.

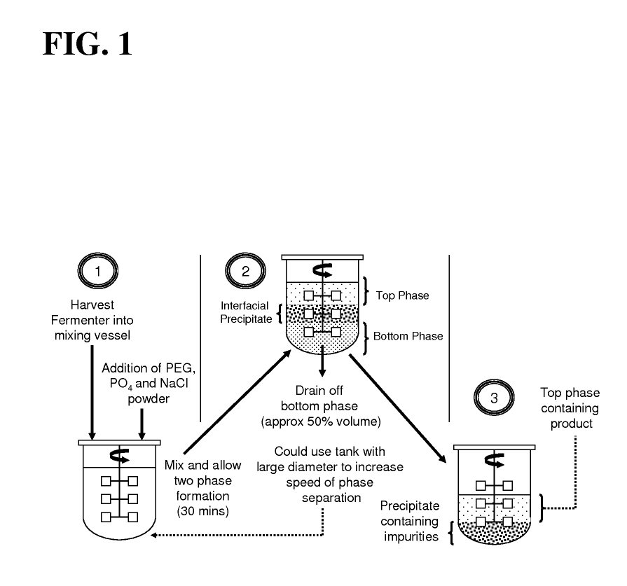

Following on, once the mixing stage of the forward extraction process has been completed, agitation of the stirred vessel may be halted, and the contents allowed to settle under the influence of gravity. Dependent upon the viscosity of the aqueous two phase system and the time permitted for the incubation period, complete phase separation may be achieved in this manner. If so, the bottom portion of the vessel contents may be drained in order to remove the bottom phase from the system, leaving only the top phase, and any precipitate which may have formed during the forward extraction process, in the vessel. If the bottom phase corresponds to the polymer rich phase, then it may be clarified using any conveniently available method such as filtration or centrifugation in order remove any precipitate carried over from the forward extraction process, before being passed onto the back extraction process. Alternatively, the top phase may be the polymer rich and therefore product containing phase. In such cases the top phase may be recovered using any conveniently available method, for example filtration or centrifugation. This embodiment is exemplified in FIG. 1. If phase separation is only partial under gravity, only a fraction of the bottom phase may be drained from the vessel. The remaining portion of bottom phase and also any precipitate which may have formed during the forward extraction process must be removed from the polymer rich top phase, using a conveniently available method, for example centrifugation. The recovered polymer rich, target protein containing top phase may then be passed on to the back extraction process.

### 2. Back Extraction

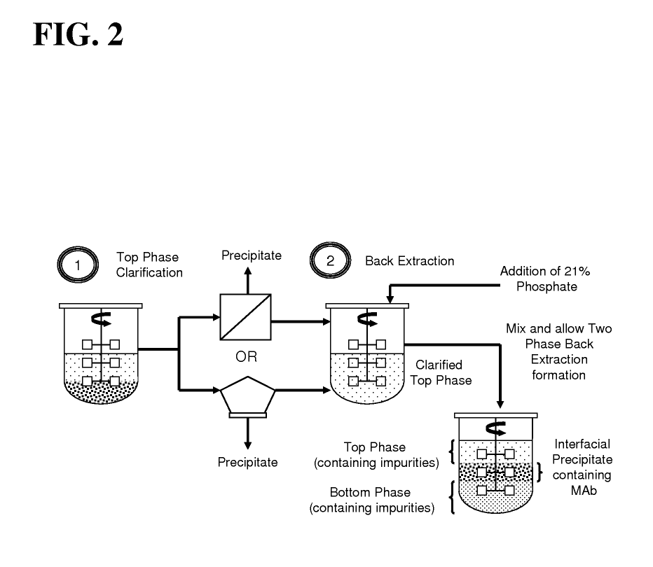

The back extraction step is performed following the forward extraction (FIG. 2). The polymer rich phase from the forward extraction is recovered and contacted with a back extraction buffer. The back extraction buffer can be a concentrated salt solution, containing a salt with an anion which is incompatible with the polymer used in the forward extraction system. The anion need not be the same as that which was used in the forward extraction system.

In one embodiment the back extraction buffer is a phosphate salt solution with a concentration of between 10% (w/w) phosphate and 40% (w/w) phosphate, for example 21% (w/w) phosphate. The back extraction buffer is made using a combination of both monobasic and dibasic phosphate. The monobasic phosphate used may be monobasic sodium phosphate (NaH₂PO₄) or monobasic potassium phosphate (KH₂PO₄). The dibasic phosphate used may be dibasic potassium phosphate (K₂HPO₄) or dibasic sodium phosphate (Na₂HPO₄). The ratio of monobasic to dibasic phosphate added may be altered to control the pH of the back extraction buffer. Increasing the amount of monobasic phosphate used, whilst decreasing the amount of dibasic phosphate added will result in a lower system pH, while increasing the amount of dibasic phosphate added and decreasing the amount of monobasic phosphate used will increase the pH of the system. The back extraction buffer should have a pH of between 3.0 and 9.0, although a relatively neutral pH is preferable, for example between pH 5.0 and 7.0, such as pH 6.

In another embodiment, the back extraction buffer is a citrate salt solution with a concentration of between 10% (w/w) citrate and 40% (w/w) citrate, for example 30% citrate. This citrate back extraction buffer can be made using sodium citrate salt.

The back extraction buffer should be mixed with the polymer rich phase from the forward extraction to form a new aqueous two phase system. The volume of back extraction buffer added should be between one and two times the volume of the polymer rich phase from the forward extraction. For example 10 mL of back extraction buffer should be added to 10 mL of polymer rich phase or 15 mL of back extraction buffer should be added to 10 mL of polymer rich phase or 20 mL of back extraction buffer should be added to 10 mL of top phase

One of ordinary skill in the art will recognise the need to optimise both the concentration of the back extraction buffer and also the volume ratio between the polymer rich phase from the forward extraction and the back extraction buffer.

The back extraction two phase system should be mixed for between 5 minutes and 30 minutes, for example 10 minutes. The aqueous two phase system may be left to incubate at room temperature for between 5 minutes and 60 minutes, for example 10 minutes. The mixing period is to maximise the surface area for mass transfer between phases, and also to ensure equilibrium is reached with regards to partitioning of components between the polymer rich phase and the salt rich phase. The incubation period is to allow for partial phase separation under gravity. Precipitation will occur during this back extraction process, which will settle (possibly requiring some assistance, for example using centrifugation) at the interface between the top and bottom phase. This precipitate will contain the majority of the target protein. Negligible amounts of the target protein will be present in the top and bottom phases of the back extraction system.

In one embodiment the back extraction process may be performed in a single agitated vessel. Following clarification of the polymer rich phase from the forward extraction to remove any precipitate which may have been present, the polymer rich phase may be held in a stirred tank to which the back extraction buffer is directly added. The back extraction aqueous two phase system may then be mixed in order to ensure equilibrium is reached and that complete partitioning of components has occurred. One of ordinary skill in the art will recognise the need to optimise the mixing process, in order to minimise mixing times within the stirred vessel. Optimisation of the mixing process will need to account for, among other things, factors such as the design of agitator(s), vessel dimensions and presence of baffles. In addition, since the target protein forms a precipitate during this stage of the process, optimisation of the mixing process must account for the need to maintain precipitate integrity and also target product quality. Once mixing has been completed, agitation of the stirred vessel may be halted, and the contents allowed to partially settle under the influence of gravity. The vessel will at this point contain a top and bottom phase containing the majority of impurities and an interfacial precipitate containing the majority of product protein. The next stage is to recover the precipitate formed during this back extraction process. This embodiment is exemplified in FIG. 2.

### 3. Precipitate Recovery and Resolubilisation

The precipitate formed during the back extraction process may be recovered by any conveniently available method, for example microfiltration or centrifugation. One skilled in the art will recognise the need to optimise the precipitate recovery process depending upon the method employed. For example the use of a filtration will require optimisation of membrane surface areas and membrane fluxes in order to minimise the rate of membrane fouling and maximise process productivity. The use of centrifugation will require optimisation in order to maximise dewatering of the precipitate whilst also minimising precipitate compaction which may reduce the ease of resuspension. Regardless, trade-offs will need to be made between desirable process attributes.

Following recovery, the precipitate may then also be washed using a suitable buffer in order to remove any residual liquid from the back extraction two phase system. This wash step is optional, but may be accomplished by contacting excess volumes of wash buffer with the recovered precipitate and removing the wash buffer using any conveniently available method, for example filtration, centrifugation or simply decanting. The precipitate may then be resuspended in a suitable buffer. The choice of resuspension buffer will depend upon a number of factors such as the characteristics of the target protein being purified as well as the requirements of the bioseparation technique to be employed following the purification method described herein. The resuspension buffer should have a pH of between 3.0 and 9.0. For example the resuspension buffer could be 60 mM sodium citrate at pH 3.4. The resuspension of the product protein precipitate should be performed within 20 hours of initial formation during the back extraction process. Preferably the resuspension process should be performed within 6 hours or less following initial precipitate formation. For example the resuspension of the precipitate should be performed within 1 hour after formation during the back extraction process.

Following resolubilisation, the product containing solution may be conditioned to a suitable pH and ionic strength, filtered to maintain sterility and placed into storage. For example when 60 mM sodium citrate buffer at pH 3.4 is used for resuspension, the resultant product containing solution may be titrated using 0.1M sodium hydroxide (NaOH) to a more neutral pH, such as pH 5.0, before being filtered using a 0.22 micron filter to remove any potential bacterial or viral contamination. This sterile filtered solution may then be stored at 4° C. for later use and/or further purification.

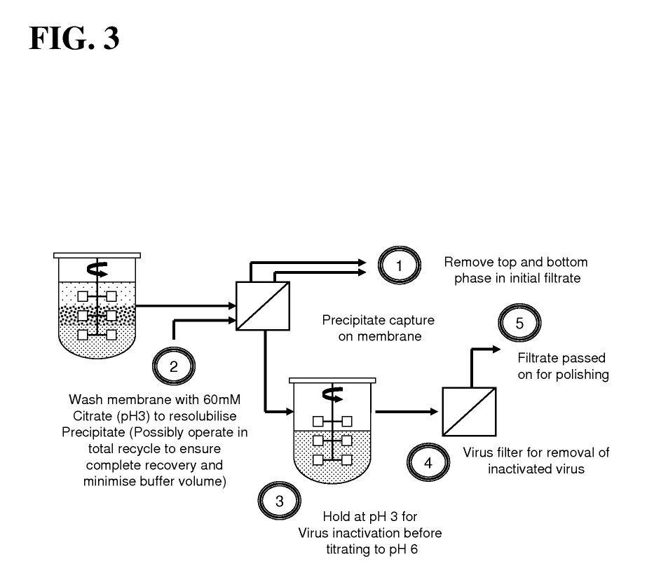

In one embodiment, in which the product is an antibody, the precipitate from the back extraction process is recovered on the surface of a microfilter. The back extraction aqueous two phase system is pumped through a 0.22 micron filter at a suitable flow rate. The antibody containing precipitate is captured on the surface of the filter membrane whilst the top and bottom phases pass through into the filtrate. The membrane may be optionally washed with a suitable buffer to remove any residual top and bottom phase. The membrane may then be washed with the resolubilisation buffer, for example 60 mM sodium citrate at pH 3.4, in order to resolubilise the precipitate, causing the antibody to emerge in the filtrate, which can then be collected in a holding vessel. This filtrate may then be passed on for further processing. One of ordinary skill in the art will recognise the need to optimise the precipitate resolubilisation in order to maximise the concentration of the antibody product, maximise the antibody yield as well as minimise buffer consumption. This will include, among other things, optimising the fluid flow through the membrane and across the membrane surface, as well as the number of times a volume of resolubilisation buffer is re-circulated through the membrane. The collected filtrate can then be, for example held at pH 3.4 in order to effect virus inactivation, before being titrated up to pH 5.0 or 6.0 using 0.1M sodium hydroxide. This conditioned product pool may then be filtered again to ensure and maintain sterility before being stored at 4° C., for later use and/or further purification. This embodiment is illustrated in FIG. 3.

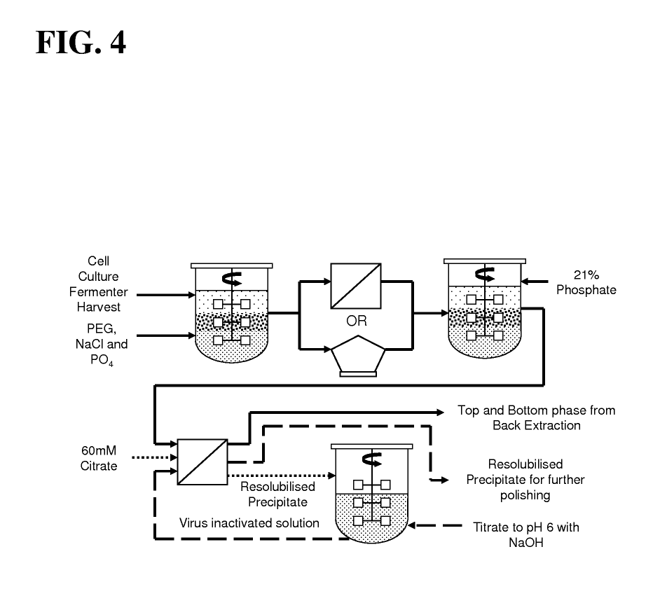

FIG. 4 collects the embodiments illustrated by FIGS. 1-3 and shows the overall process flow sheet and the equipment requirements for this ATPE augmented precipitation process at preparative or manufacturing scale. Overall three mixing vessels will be required in order to perform the forward extraction, back extraction and finally, if necessary, virus inactivation. The first vessel, used to perform the forward extraction may also be used to perform the virus inactivation provided that clean in place (CIP) and sterilise in place (SIP) procedures may be performed in time. In such a case the entire process may be performed using only two mixing vessels. Either a centrifuge or filtration unit will be required in order to recover the top phase from the forward extraction and then finally a filtration unit will be required in order to recover and resolubilise the precipitate and also for virus removal. A centrifuge may also be used to recover the product containing precipitate, which may then be transferred to a mixing vessel for resolubilisation.

In one embodiment of this invention, the entire method may be employed in place of Protein A affinity chromatography for primary capture of antibody. The resolubilised precipitate may then be applied to unit operations which would be typically found to subsequently after protein A affinity chromatography in a mAb purification process. For example the resolubilised precipitate may be run on a cation exchange chromatography column packed with for example CAPTO™ S, in bind and elute mode. The antibody containing eluate from the cation exchange step may then be applied to an anion exchange column packed with for example CAPTO™ Q, in flow through mode with the antibody containing flow through collected.

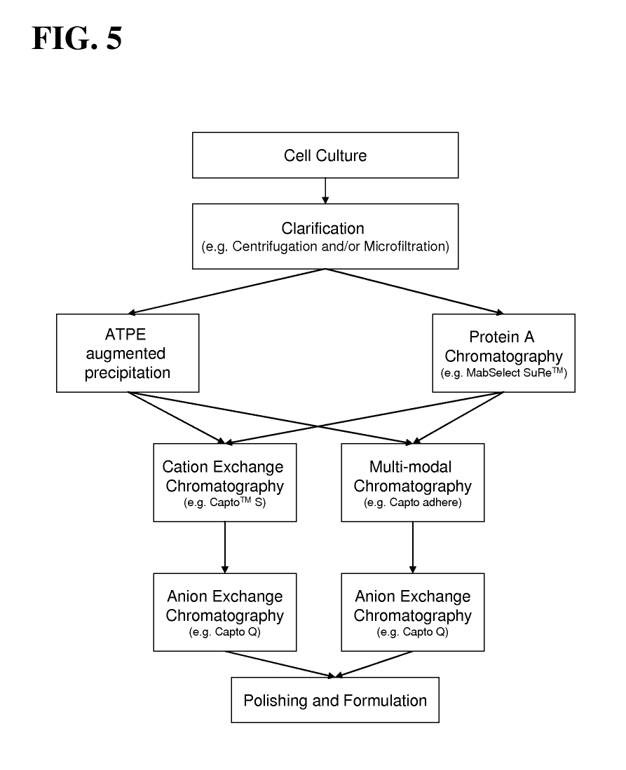

In another embodiment, the resolubilised precipitate containing the antibody may be applied to a multi-modal chromatography column packed with for example CAPTO™ Adhere, in flow through mode. The antibody containing flow through may then be applied to an anion exchange column packed with for example CAPTO™ Q, in flow through mode with the antibody containing flow through collected. These embodiments are collectively illustrated in FIG. 5.

It is noted that all embodiments of this invention can be employed on any scale. For example, the present invention can be applied to large scale antibody production, in which the antibody is to be purified from tens of thousands of litres of cell culture supernatant. In another example the present invention may be employed on a much smaller scale, for example in bench top scale operations, in which antibodies are purified from several litres or less of cell culture supernatant.

### EXAMPLES

The following examples serve to illustrate embodiments of the present invention. These examples are intended to demonstrate techniques which the present inventors have found to work well in practising the present invention. Hence these examples are detailed so as to provide those of ordinary skill in the art with a complete disclosure and description of the ways in which the methods of this invention may be performed. The following Examples are intended to be exemplary only and changes, modification and alterations can be employed to the conditions described herein, without departing from the scope of the invention.

### Example 1

### Primary Capture and Purification of MAb Using ATPE Assisted Precipitation

Chinese Hamster Ovary (CHO) cell culture supernatant was generated in house by GE Healthcare Biosciences (Uppsala, Sweden) through the culturing of cells from a cell line obtained from Polymun Scientific (Vienna, Austria). The cell culture supernatant was obtained by harvesting of the cell culture followed by centrifugation and depth filtration in order to remove whole unlysed cells. The cell culture supernatant was found to contain a monoclonal human IgG antibody, denoted Antibody A, at a titre of less than 1 g/L. This supernatant was concentrated by approximately ten fold using ultrafiltration giving a final mAb concentration of 4.5 g/L. This cell culture supernatant was sterile filtered using a 0.22 micron microfilter before being stored at 4° C. prior to being subjected to the ATPE assisted precipitation process.

Polyethylene glycol (PEG), with molecular weights of 1500 and 6000, along with Tris(hydroxymethyl)aminomethane and 3-bromopropyl trimethyl ammonium bromide were obtained in the form of powders from Sigma-Aldrich. Sodium phosphate monobasic monohydrate (NaH₂PO₄.H₂O), potassium phosphate dibasic trihydrate (K₂HPO₄.3H₂O), citric acid monohydrate (C₆H₈O₇.H₂O), tri-sodium citrate (Na₃C₆H₅O₇) and sodium hydroxide (NaOH) were also obtained in the form of powders and were purchased from Merck Chemicals Ltd (Nottingham, UK). Sodium chloride (NaCl) was obtained from VWR International Inc.

### Aqueous Two Phase Extraction

I. Forward Extraction 30 g ATPE forward extraction systems were generated by adding appropriate amounts of PEG 1500, K₂HPO₄.3H₂O, NaH₂PO₄.H₂O and NaCl powders directly to the cell culture supernatant feed so as to give a final system composition of 15% PEG 1500, 14% Phosphate and 12% NaCl. More specifically, 4.50 g of PEG 1500, 2.44 g of K₂HPO₄. 3H₂O, 2.66 g of NaH₂PO₄.H₂O and 3.60 g of NaCl were added to 17.7 mL of cell culture supernatant to form the ATPE forward extraction system.

Systems were formed in 50 mL FALCON™ tubes (BD Biosciences), with system mixing accomplished by placing the FALCON™ tubes onto a rocking platform shaker (custom manufactured at GE Healthcare). Powders were added sequentially with NaCl added first, followed by PEG 1500 and finally the K₂HPO₄. 3H₂O and NaH₂PO₄.H₂O. The monobasic and dibasic phosphates were added so as to give the desired system phosphate mass percentage whilst the pH of the system was controlled by altering the ratio between the mass of monobasic and dibasic salt added. In this case, the actual ratio between the amounts of monobasic and dibasic phosphate added, resulted in a forward extraction system with a pH of 6.0. Sufficient time was allowed between the additions of powders so as to ensure complete dissolution of the previously added component before the introduction of the next. Powders were usually completely dissolved after approximately 10 minutes. Precipitation was observed following addition of the phosphate salts

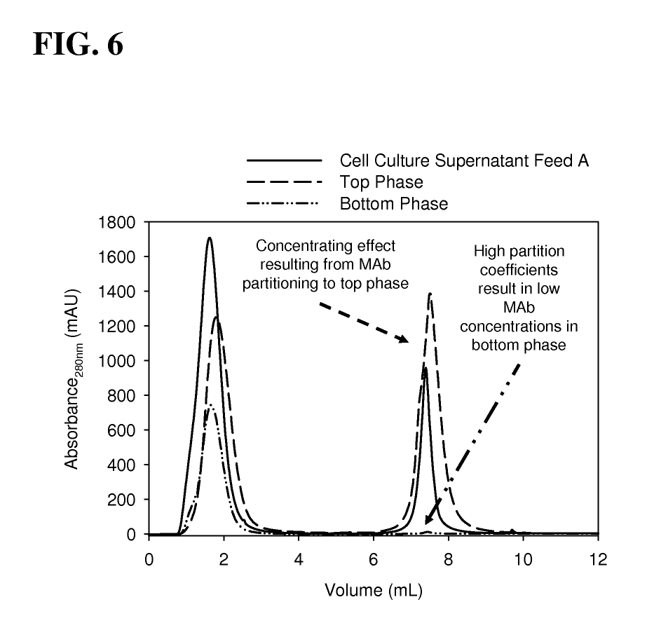

Following addition and dissolution of all powders systems were mixed for a further 60 minutes before being left to settle under gravity for 30 minutes in order to effect phase separation. Only partial phase separation was achieved, possibly due to the precipitate in the aqueous two phase system affecting the settling velocity of the bottom salt rich phase. Systems were centrifuged at 3000 rpm for 30 mins, using an Eppendorf 5810R (Eppendorf, Hamburg, Germany) centrifuge in order to obtain complete phase separation. The forward extraction system was observed to be composed of three discreet phases, settled one on top of the other, following centrifugation; a top polymer rich phase, an interfacial precipitate and a salt rich bottom phase. The top and bottom phases were then carefully separated and the volumes determined Samples were taken and analysed in order to determine the concentration of antibody in each phase. FIG. 6 is an example of the results obtained from this analysis. It shows a comparison of Chromatograms from Protein A analyses (using a MABSELECT SURE™ 1 mL HITRAP™ column) of top and bottom phases of a forward extraction aqueous two phase system applied to cell culture supernatant feed, containing antibody A and denoted “cell culture supernatant feed A”. The chromatogram for the Protein A analysis of this feed is also included for comparison. Antibody A is a monoclonal IgG. Peak 1, with a column retention of approximately 1.5 to 2 mL in all chromatograms, corresponds to unbound UV₂₈₀ adsorbing impurities present in the sample. Peak 2 with a retention of approximately 7.5 mL in all chromatograms, corresponds to bound antibody A. The chromatograms shown in FIG. 6 indicate a high level of antibody A partitioning to the top phase of the forward extraction system with little to no antibody present in the bottom phase. Mass balances based on the integration of peaks shows partition coefficients of greater than 100. A mass balance also showed a yield of greater than 100% in the top phase. This indicates that the presence of PEG affects the UV absorbent properties of the antibody in some way (blank systems of PEG showed no absorbance at UV₂₈₀ₙₘ). Due to this it is difficult to accurately determine the MAb content of the interfacial precipitate which was found to form during the forward extraction.

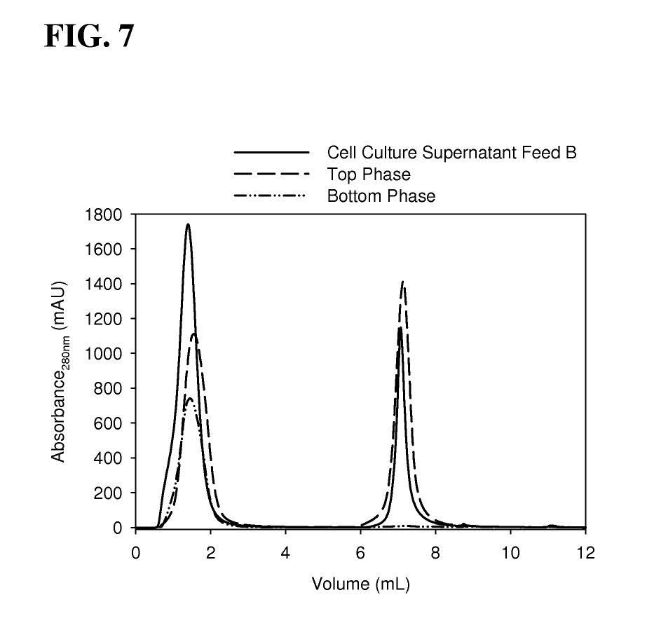

FIG. 7 shows a comparison of Chromatograms similar to that shown in FIG. 6, albeit with a different feed material, this time a CHO cell culture feed supernatant, containing antibody B and denoted “cell culture supernatant feed B”. The chromatogram for the protein A analysis of this feed is included for comparison. Antibody B is also a monoclonal IgG. As with the results shown in FIG. 6, FIG. 7 shows a high level of antibody B partitioning to the top phase of the forward extraction system with little to no antibody present in the bottom phase. Mass balances based on the integration of peaks shows partition coefficients of approximately 70. Again mass balances showed a yield of greater than 100% in the top phase, apparently due to the presence of PEG affecting the UV absorbent properties of the antibody.

II. Back Extraction Back extraction was performed by taking the top phase from the forward extraction system and adding a back extraction buffer in order to generate a new two phase system. The back extraction buffer utilised was a phosphate buffer solution, made using K₂HPO₄. 3H₂O and NaH₂PO₄.H₂O, added to give a phosphate concentration of 21% (w/w) and in a ratio as to give a pH of 6.0.

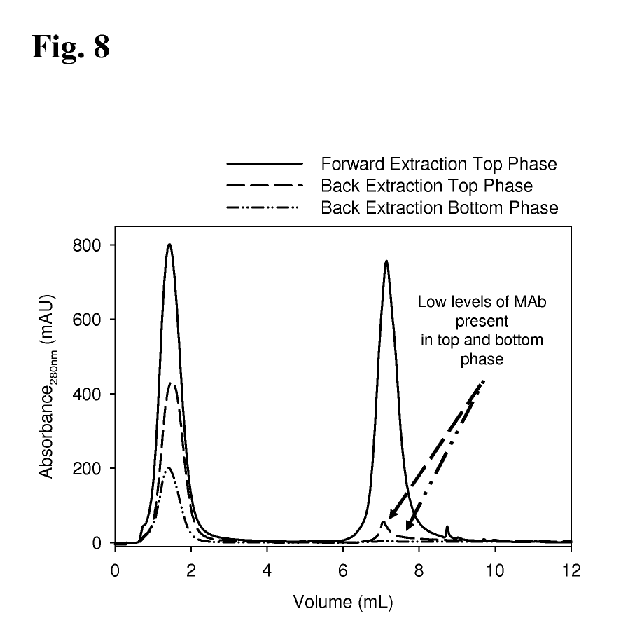

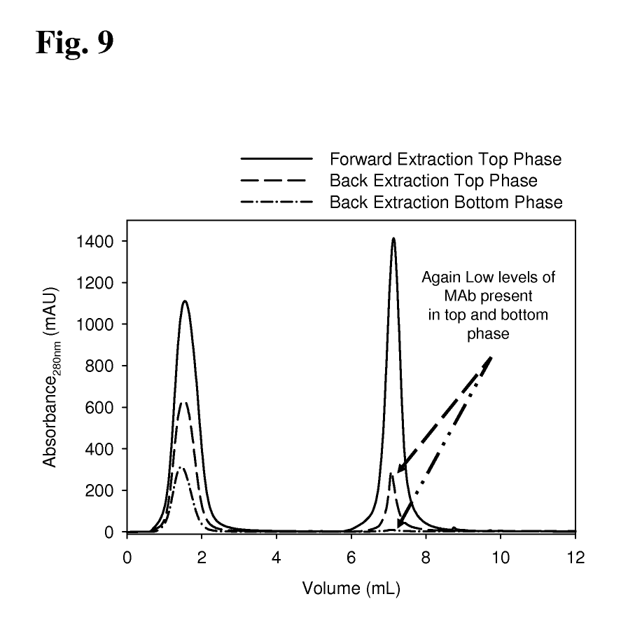

Back extraction systems were formed in 50 mL FALCON™ tubes, with back extraction buffer added to the top phase, recovered from the forward extraction, in a volume ratio (top phase:bottom phase) of 1:2. Back extraction systems were mixed in the same manner as the forward extraction systems, using the rocking platform, for approximately 10 minutes. Precipitation was observed during the mixing process. Back extraction systems were then allowed to settle under gravity for 15 minutes, before being centrifuged at 3000 rpm for 10 minutes to ensure complete phase separation using an Eppendorf 5810R (Eppendorf, Hamburg, Germany) centrifuge. The back extraction system was observed to be composed of three discrete phases, settled one on top of the other, following centrifugation; a top polymer rich phase, an interfacial precipitate and a salt rich bottom phase. Samples of the top and bottom phases were taken for analysis. FIGS. 8 and 9 show the chromatograms obtained from a Protein A affinity chromatography analysis of the top and bottom phases obtained from the back extraction systems. FIG. 8 shows a comparison of Chromatograms from Protein A analyses of top and bottom phases of a back extraction aqueous two phase system performed on the top phase obtained from the forward extraction on cell culture supernatant feed A. The chromatogram from the Protein A analysis of the top phase from the forward extraction on CHO cell culture supernatant A containing antibody A is also included for comparison. Peak 1, with a column retention of approximately 1.5 to 2 mL in all chromatograms, corresponds to unbound UV₂₈₀ adsorbing impurities present in the sample. Peak 2 with a retention of approximately 7.5 mL in all chromatograms, corresponds to bound antibody A. The low concentrations of antibody A in top and bottom phase indicate that the majority of antibody has collected in the interfacial precipitate which was found to form during the back extraction. Based on antibody concentrations in top and bottom phase and that present in the top phase from the forward extraction, calculations indicate an antibody yield of between 85 and 90% in the precipitate. A mass balance using peak integration also indicates that this precipitate contains a low level of impurities.

FIG. 9 shows a comparison of Chromatograms, similar to FIG. 8, of top and bottom phases of a back extraction aqueous two phase system performed on the top phase obtained from the forward extraction on cell culture supernatant feed B. The chromatogram from the Protein A analysis of the top phase from the forward extraction on CHO cell culture supernatant B containing antibody B is also included for comparison. Calculations indicate an antibody yield of between 85 and 90% in the precipitate. A mass balance using peak integration also indicates that this precipitate contains a low level of impurities.

### Precipitate Recovery and Resolubilisation

The top and bottom phases of the back extraction system were carefully removed using a pipettor (VWR International Inc.) leaving only the precipitate in the FALCON™ tube. 60 mM sodium citrate buffer at pH 3.4 was placed into the FALCON™ tube with the precipitate and mixed used a RX3 vortex mixer (VELP Scientifica, Italy). The precipitate resolubilised almost instantaneously upon mixing in this manner. Precipitate resolubilisation was performed at room temperature. This resolubilisation procedure was performed within 1 hour of initial precipitate formation during the back extraction process.

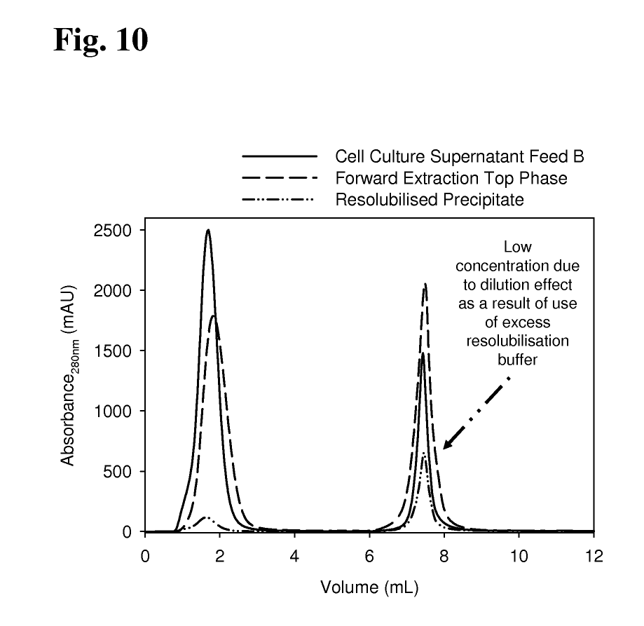

After resolubilisation, the antibody containing sample was incubated at room temperature for 60 minutes for virus inactivation. The sample was then slowly titrated up to pH 5.0 using 0.1M NaOH. Samples were then analysed for antibody content, using Protein A chromatography. FIG. 10 shows an example of the results obtained from this analysis. Specifically it shows a comparison of Chromatograms from Protein A analyses of the top phase obtained from the forward extraction performed on cell culture supernatant feed B, containing antibody B and of the resolubilised precipitate formed in and recovered from the back extraction aqueous two phase system. The chromatogram from the CHO cell culture feed supernatant, containing antibody B and denoted “cell culture supernatant feed B” is also included for comparison. Peak 1, with a column retention of approximately 1.5 to 2 mL in all chromatograms, corresponds to unbound UV₂₈₀ adsorbing impurities present in the sample. Peak 2 with a retention of approximately 7.5 mL in all chromatograms, corresponds to bound antibody B. FIG. 10 shows the increase of MAb purity as it moves into the top phase during forward extraction and then into the precipitate during back extraction. The low concentration of MAb in the re-solubilised precipitate sample is due to the use of excess re-solubilisation buffer used in this particular experiment. The relative heights of the peaks indicate that this ATPE augmented precipitate process has afforded a significant level of purification.

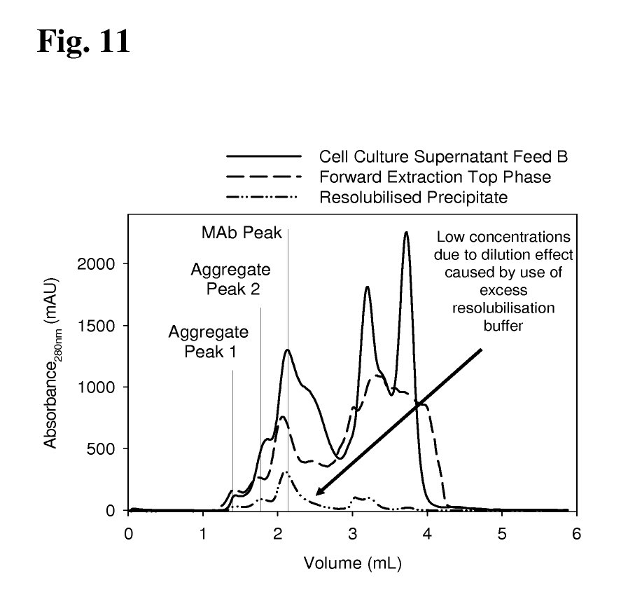

An analysis of the product quality and aggregate content throughout the ATPE augmented precipitation process was performed using size exclusion chromatography. As an example, FIG. 11 shows a comparison of Chromatograms from size exclusion chromatography analyses (using a SUPERDEX™ 200 10/30 column) of the cell culture supernatant feed B, containing antibody B, the top phase from the forward extraction aqueous two phase system performed on cell culture supernatant feed B and the resolubilised precipitate subsequently formed in and recovered from the back extraction aqueous two phase system. The initial feed was found to have an aggregate content of approximately 16%. The aggregate content in the final re-solubilised precipitate was calculated to be approximately 20%. This is comparable to what would normally be achieved using Protein A as a primary capture step. The size exclusion analysis further indicates the significant level of purification achieved using this current process.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method of recovering and purifying a protein from a multi-component mixture, the method comprising of:
a. Adding phase forming components including a polymer, a salt containing an incompatible anion and a partition mediating salt to said multi-component mixture;
b. Mixing and completely dissolving aforementioned phase forming components to form a forward extraction aqueous two phase system;
c. Recovering the polymer rich phase;
d. Contacting said polymer rich phase with back extraction buffer to form a back extraction aqueous two phase system, including an interfacial precipitate containing the protein;
e. Recovering the interfacial precipitate from the two phase system; and
f. Optionally, resuspending the precipitate in a resuspension buffer.

2. The method of claim 1, wherein the polymer is selected from Polyethylene glycol (PEG) and ethylene oxide-propylene oxide (EOPO), the incompatible anion is selected from strongly hydrated anions including phosphate, citrate and sulphate and the partition mediating salt is selected from less strongly hydrated anions, including NaCl and potassium iodide (KI)

3. The method of claim 2, wherein the polymer used is polyethylene glycol (PEG), the incompatible anion is phosphate and the partition mediating salt is NaCl.

4. The method of claim 3, wherein the concentration of PEG in the two phase system is between 12% and 20% (w/w), the concentration of Phosphate is between 9% and 19% (w/w) and the concentration of NaCl in the forward extraction aqueous two phase system is between 4% and 12% (w/w).

5. The method of claim 4, wherein the concentration of PEG in the forward extraction system is 15% (w/w), the concentration of phosphate is 14% (w/w) and the concentration of NaCl is 12% (w/w).

6. The method of claim 3, wherein the PEG added to the feed has a molecular weight of between 1,450 Da and 6,000 Da, such as a molecular weight of 1,500 Da.

7-8. (canceled)

9. The method of claim 1, step (a), wherein the phase forming components are added to the feed in the form of powders.

10. The method of claim 1, wherein the pH of the forward extraction aqueous two phase system is between 3.0 and pH 9.0, such as a system pH of 6.0.

11. The method of claim 1, wherein following complete dissolution of phase forming components, the forward extraction system is incubated for between 10 minutes and 24 hours, such as for 30 minutes.

12. (canceled)

13. The method of claim 1, wherein the polymer rich phase of step (c) is recovered by:
a. Gravity settling of the two phase system allowing for complete phase separation; followed by
b. Draining of bottom phase with or without any interfacial precipitate or aspirating the top phase.

14. The method of claim 1, wherein the polymer rich phase of step (c) is recovered by:
a. Centrifugation of the two phase system; followed by
b. Removing the bottom phase and any interfacial precipitate or aspirating the top phase.

15. The method of claim 1, wherein the back extraction buffer is a concentrated salt solution.

16. The method of claim 15, wherein the anion of the salt composing the back extraction buffer is selected from citrate, phosphate and sulphate.

17. The method of claim 16, wherein the back extraction buffer is phosphate solution, with a concentration of between 10% (w/w) and 40% (w/w).

18. The method of claim 17, wherein the back extraction buffer has a pH of between 3.0 and 9.0, such as a pH of 6.0.

19. The method of claim 1, wherein the volume of back extraction buffer contacted with the polymer rich phase from the forward extraction is between one and two times the volume of the polymer rich phase from the forward extraction.

20. The method of claim 1, wherein following mixing, the back extraction aqueous two phase system is incubated for between 5 minutes and 15 minutes, such as for 10 minutes.

21. The method of claim 1, wherein the precipitate formed during the back extraction is recovered and resuspended by:
a. Filtration of the back extraction system;
b. Capturing the precipitate on the membrane surface; and
c. Flushing the membrane with resuspension buffer to resuspend the antibody precipitate and collecting the filtrate.

22. The method of claim 21, wherein the resuspension buffer is re-circulated across and through the membrane to affect precipitate re-solubilisation.

23. The method of claim 1, wherein the precipitate formed during the back extraction is recovered and resolubilised by:
a. Centrifugation of the back extraction aqueous two phase system;
b. Removal of liquid top and bottom phases; and
c. Resuspending the precipitate in resuspension buffer.

24-26. (canceled)

27. The method of claim 1, wherein the recovery and resuspension of the precipitate is performed within 20 hours, such as within 6 hours or within 1 hour, of formation during the back extraction process.

28-29. (canceled)
